(19) United States
(12) Patent Application Publication
(10) Pub. No.: US 2022/0399092 A1
(43) Pub. Date: Dec. 15, 2022

(54) AI-ENABLED HEALTH PLATFORM

(71) Applicant: Rajant Health Inc., Malvern, PA (US)

(72) Inventors: Robert J Schena, Malvern, PA (US); Emma K. Murray, Malvern, PA (US); Giana J. Schena, Malvern, PA (US); Muthukumaran Chandrasekaran, Malvern, PA (US)

(21) Appl. No.: 17/806,477

(22) Filed: Jun. 10, 2022

Related U.S. Application Data
(60) Provisional application No. 63/209,307, filed on Jun. 10, 2021.
(60) Provisional application No. 63/209,298, filed on Jun. 10, 2021.
(60) Provisional application No. 63/209,291, filed on Jun. 10, 2021.

(51) Int. Cl. G16H 20/10; G16H 10/40; G16H 50/20; G16H 50/50; G16H 70/60

(52) U.S. Cl. CPC G16H 20/10; G16H 10/40; G16H 50/20; G16H 50/50; G16H 70/60

(57) ABSTRACT

An artificial intelligence-enabled health ecosystem that leverages physiological data (captured, for example, by wearable health monitoring devices), medical history data (e.g., including biofluid data captured by biofluid analyzers), contextual information relevant to health outcomes, and genetic data (captured, for example, by genetic analyzers) to identify correlations in disparate health data, so that inferences can be drawn, health outcomes can be better anticipated and managed, and targeted drugs can be developed.

### CROSS-REFERENCE TO RELATED APPLICATIONS

This application claims priority to U.S. Prov. Pat. Appl. No. 63/209,307, filed Jun. 10, 2021; U.S. Prov. Pat. Appl. No. 63/209,298, filed Jun. 10, 2021; and U.S. Prov. Pat. Appl. No. 63/209,291, filed Jun. 10, 2021. The subject matter of this application is also related to the subject matter of co-pending U.S. patent application Ser. No. 17/833,842, filed Jun. 6, 2022, and U.S. patent application Ser. No. 17/806,475, filed contemporaneously herewith. All of the aforementioned applications are hereby incorporated by reference.

### FEDERAL FUNDING

None

### BACKGROUND

Modern technology captures a variety of information about the health of individuals. Wearable devices capture physiological data. Biofluid analyzers capture biofluid data. Genetic analyzers capture genetic data. Electronic health records systems store medical records. Those health records systems and other computer systems store contextual information (e.g., demographic information, age, mood, etc.) relevant to health outcomes.

Physiological data may be indicative of a medical event. Biofluid data may be indicative of a disease. Combining medical records and physiological data with genetic data can be used to better identify drugs specifically targeted for individuals. Artificial intelligence and machine learning can be used to identify correlations in disparate health data so that inferences can be drawn, health outcomes can be better anticipated and managed, and targeted drugs can be developed.

Using convention health systems, however, all of that disparate medical data is siloed in separate computer systems.

Accordingly, there is a need for an artificial intelligence-enabled health ecosystem that leverages physiological data (captured, for example, by wearable health monitoring devices), medical history data (e.g., including biofluid data captured by biofluid analyzers), contextual information relevant to health outcomes, and genetic data (captured, for example, by genetic analyzers) to identify correlations in disparate health data so that inferences can be drawn, health outcomes can be better anticipated and managed, and targeted drugs can be developed.

### SUMMARY

Disclosed is an artificial intelligence-enabled health ecosystem that leverages physiological data (captured, for example, by wearable health monitoring devices), medical history data (e.g., including biofluid data captured by biofluid analyzers), contextual information relevant to health outcomes, and genetic data (captured, for example, by genetic analyzers) to identify correlations in disparate health data, so that inferences can be drawn, health outcomes can be better anticipated and managed, and targeted drugs can be developed.

Also disclosed is a personalized, genetics-based drug discovery process that identifies a drug to treat a disease in individuals having a common attribute by repeatedly partitioning a group of individuals having a disease to select a subgroup of individuals having a common attribute and, for each selected subgroup, detecting physiological or medical test anomalies that are more prevalent in the selected subgroup than in a control group, identifying genetic anomalies affecting gene(s) that are more prevalent in the selected subgroup than in the control group, identifying a disease signature by identifying the anomalies that are more prevalent in the selected subgroup than in previously selected subgroups of individuals having the disease, identifying physiological functions affected by the physiological anomalies or medical test anomalies, identifying biological functions affected by the genes having the genetic anomalies, ranking the potential nodal points (from among the genes having genetic anomalies) that are most likely to have caused the largest number of the identified genetic anomalies, identifying (based on the affected physiological functions and the affected biological functions) the disease driver (from among the potential nodal points) most likely to have caused the genetic anomalies, and identifying a drug that binds to a protein made by the disease driver.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1 is a diagram of an architecture of an artificial intelligence-enabled health ecosystem according to an exemplary embodiment.

FIG. 2A is a view of a wearable health monitoring device according to an exemplary embodiment.

FIG. 2B is another view of the wearable health monitoring device of FIG. 2A according to an exemplary embodiment.

FIG. 2C is a view of the sensor modules of the wearable health monitoring device of FIGS. 2A and 2B according to an exemplary embodiment.

FIG. 2D is another view of the sensor modules of the wearable health monitoring device of FIGS. 2A and 2B according to an exemplary embodiment.

FIG. 2E is another view of the sensor modules of the wearable health monitoring device of FIGS. 2A and 2B according to an exemplary embodiment.

FIG. 2F is another view of the sensor modules of the wearable health monitoring device of FIGS. 2A and 2B according to an exemplary embodiment.

FIG. 2G is a view of sensor boards of the wearable health monitoring device of FIGS. 2A and 2B according to an exemplary embodiment.

FIG. 2H is another view of the sensor boards of the wearable health monitoring device of FIGS. 2A and 2B according to an exemplary embodiment.

FIG. 2I is a view of the sensor module connection ports of the wearable health monitoring device of FIGS. 2A and 2B according to an exemplary embodiment.

FIG. 2J is another view of the sensor module connection ports of the wearable health monitoring device of FIGS. 2A and 2B according to an exemplary embodiment.

FIG. 2K is a block diagram of the wearable health monitoring device according to exemplary embodiments.

FIG. 3A is a diagram of the artificial intelligence-enabled health ecosystem according to an exemplary embodiment.

FIG. 3B is a diagram of a process for generating a biofluid model for identifying biofluid signals according to an exemplary embodiment.

FIG. 3C is a diagram of a process for generating biofluid thresholds for generating biofluid health inferences according to an exemplary embodiment.

FIG. 3D is a diagram of a process for generating calibration parameters for digital signal processing according to an exemplary embodiment.

FIG. 3E is a diagram of a process for generating a physiological model for identifying physiological signals according to an exemplary embodiment.

FIG. 3F is a diagram of a process for generating physiological thresholds for generating physiological health inferences according to an exemplary embodiment.

FIG. 3G is a diagram of a process for generating a normalization algorithm for normalizing genetic sequence data according to an exemplary embodiment.

FIG. 4A is a block diagram of a local computing device according to exemplary embodiments.

FIG. 4B is a block diagram of a type erasure process according to an exemplary embodiment.

FIG. 4C is another block diagram of the type erasure process of FIG. 10B according to an exemplary embodiment.

FIG. 5A is a diagram of data transformation modules executed by the local computing device according to exemplary embodiments.

FIG. 5B is a diagram of other data transformation modules executed by the local computing device according to exemplary embodiments.

FIG. 5C is a diagram of the data transformation modules of FIG. 5B executed by a wearable health monitoring device according to exemplary embodiments.

FIG. 6 is a flowchart of a personalized drug discovery process according to exemplary embodiments.

FIG. 7A is a diagram of the personalized drug discovery process according to an exemplary embodiment.

FIG. 7B is a diagram continuing the personalized drug discovery process of FIG. 7A according to an exemplary embodiment.

FIG. 7C is a diagram continuing the personalized drug discovery process of FIGS. 7A and 7B according to an exemplary embodiment.

FIG. 7D is a diagram continuing the personalized drug discovery process of FIGS. 7A through 7C according to an exemplary embodiment.

FIG. 8 is a diagram of a novel annotation process according to an exemplary embodiment.

FIG. 9A is a diagram of process for modeling the cellular environment in disease according to an exemplary embodiment.

FIG. 9B is another diagram of the process for modeling the cellular environment in disease of FIG. 9A according to an exemplary embodiment.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

### System Architecture

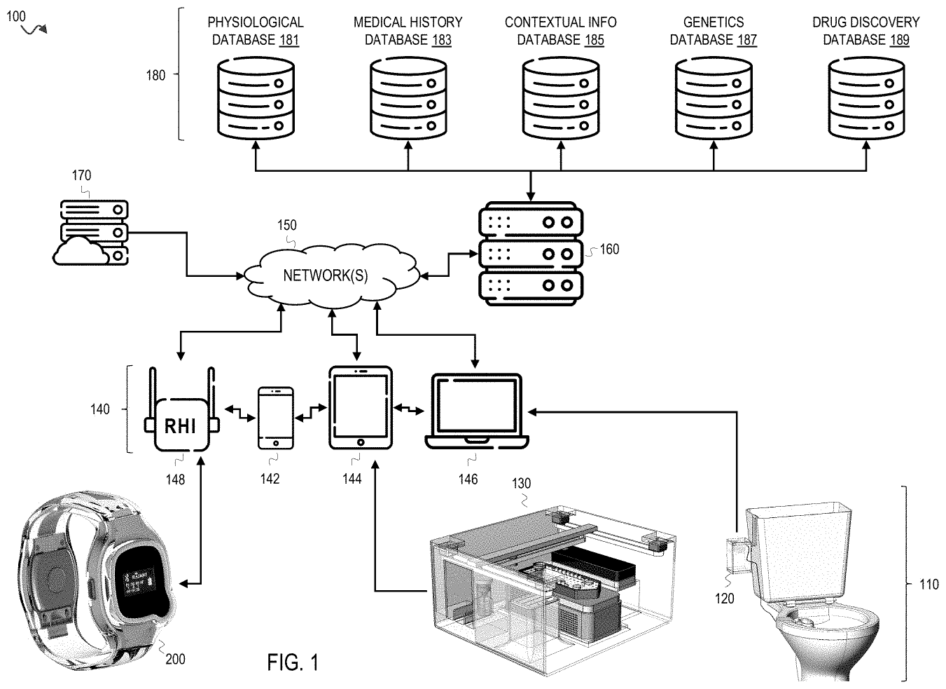

FIG. 1 is a diagram of an architecture of an artificial intelligence-enabled health ecosystem 300 according to an exemplary embodiment.

As shown in FIG. 1, the architecture 100 includes data acquisition devices 110 that communicate with a server 160 via local computing devices 140 and one or more computer networks 150. The server 160 stores data in non-transitory computer readable storage media 180 and may also receive data from third-party computer systems 170 (e.g., electronic health records systems) via the computer network(s) 150. In the embodiment of FIG. 1, the computer readable storage media 180 includes a physiological database 181, a medical history database 183, a contextual information database 185, a genetics database 187, and a drug discovery database 189. Those databases may be any collection of information stored in any hardware storage device and any number hardware storage devices.

The data acquisition devices 110 may include a wearable health monitoring device 200 (for example, the modular wristband and sensor system 200 or 300 described in co-pending U.S. patent application Ser. No. 17/806,475), a biofluid analyzer 120 (for example as described in co-pending U.S. patent application Ser. No. 17/833,842), a genetic sequencer 130, etc. As described below, each data acquisition device 110 may include multiple sensors.

The biofluid analyzer 120 may be any device capable of analyzing biofluid to identify biological markers of changing health and disease states. For example, the biofluid analyzer 120 may capture biofluid and dispense the captured biofluid (e.g., a predetermined amount of biofluid) into a chemically coated disposable cartridge. The biofluid and the chemical coating may initiate chemical reactions that cause color changes in the disposable cartridge that are indicative of biological markers. The biofluid analyzer 120 may then measure those color changes (e.g., using a spectrometer) and output data indicative of those biological markers to a local computing device 140.

The genetic sequencer 130 may be any device capable of revealing the presence, quantity, and sequence of ribonucleic acid (RNA) and/or in deoxyribonucleic acid (DNA). For example, the genetic sequencer 130 may collect a genetic sample (e.g., blood, urine, saliva, etc.), isolate RNA, create complementary DNA (cDNA), and sequence the RNA.

In preferred embodiments, the data acquisition devices 110 wirelessly communicate with the local computing devices 140 directly (e.g., using Zigbee, Bluetooth, Bluetooth Low Energy, ANT, etc.) or via a local area network (e.g., a Wi-Fi network). In other embodiments, a data acquisition devices 110 may transfer data using a wired connection (e.g., a USB cable) or by storing data in a removable storage device (e.g., a USB flash memory device, a microSD card, etc.) that can be removed and inserted into a local computing device 140.

The local computing devices 140 may include any hardware computing device having one or more hardware computer processors that perform the functions described herein. For example, the local computing devices 140 may include smartphones 142, tablet computers 144, personal computers 146 (desktop computers, notebook computers, etc.), etc. The local computing devices 140 may also include dedicated processing devices 148 (installed, for example, in hospitals or other clinical settings) that form local access points to wirelessly receive data from wearable health monitoring devices 200 and/or other data acquisition devices 110.

As described in detail below, the local computing devices 140 receive and process data from the data acquisition devices 110 and output the processed data to the server 160 via the one or more networks 150 (e.g., local area networks, cellular networks, the Internet, etc.). In some embodiments, the local computing devices 140 wirelessly communicate with each other, either via a local area network 150 or using direct, wireless communication (e.g., via Bluetooth, Zigbee, etc.) to form a mesh network. Accordingly, in some embodiments, a data acquisition device 110 may output data to a child data acquisition device 110, which forwards that data to a parent data acquisition device 110 and forwards the data to the server 160. The server 160 may be any hardware computing device having one or more hardware computer processors that perform the functions described herein.

### Wearable Health Monitoring Device 200

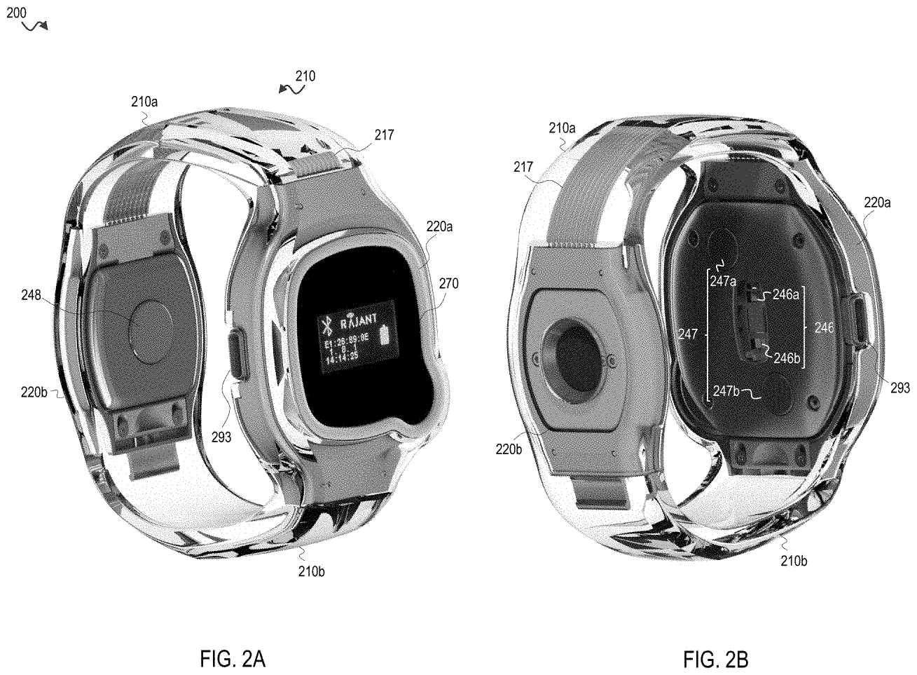

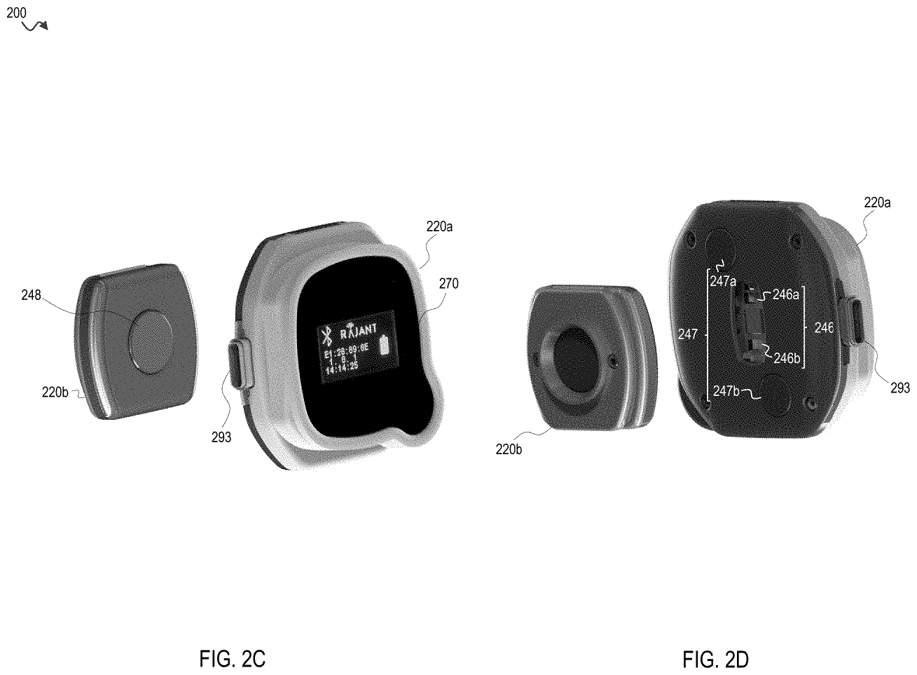

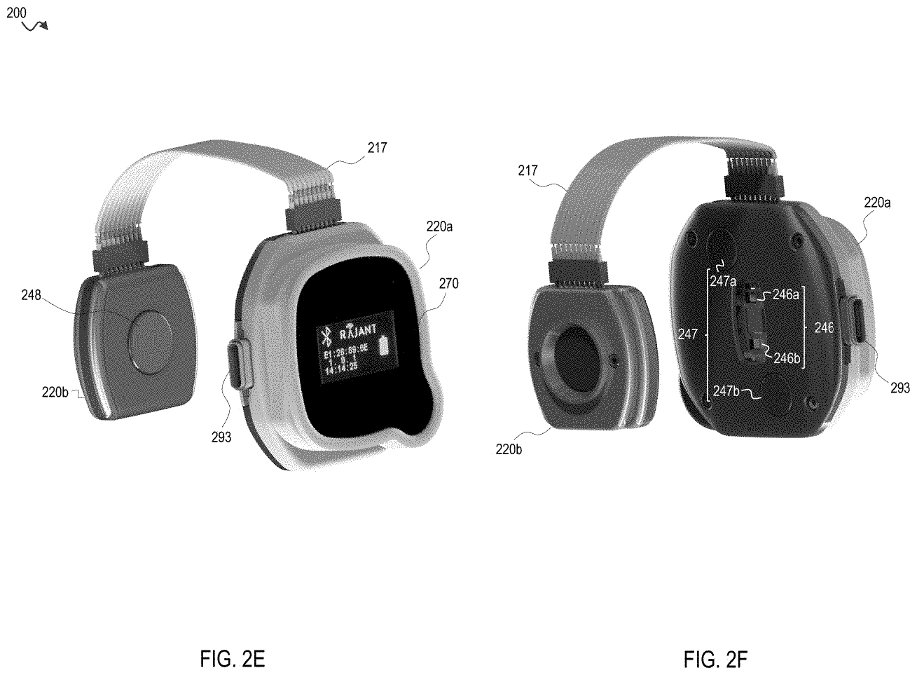

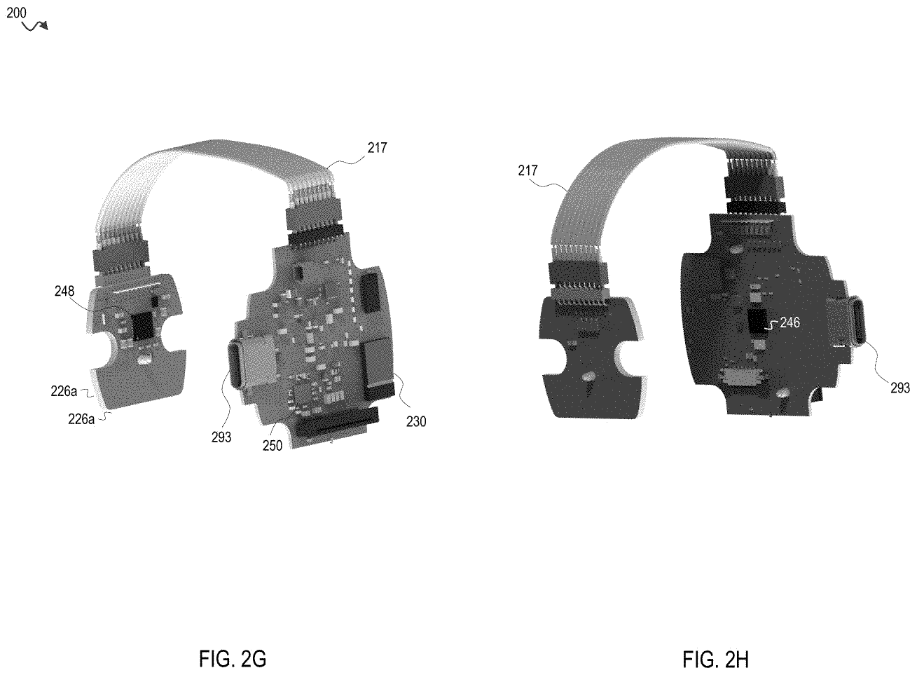

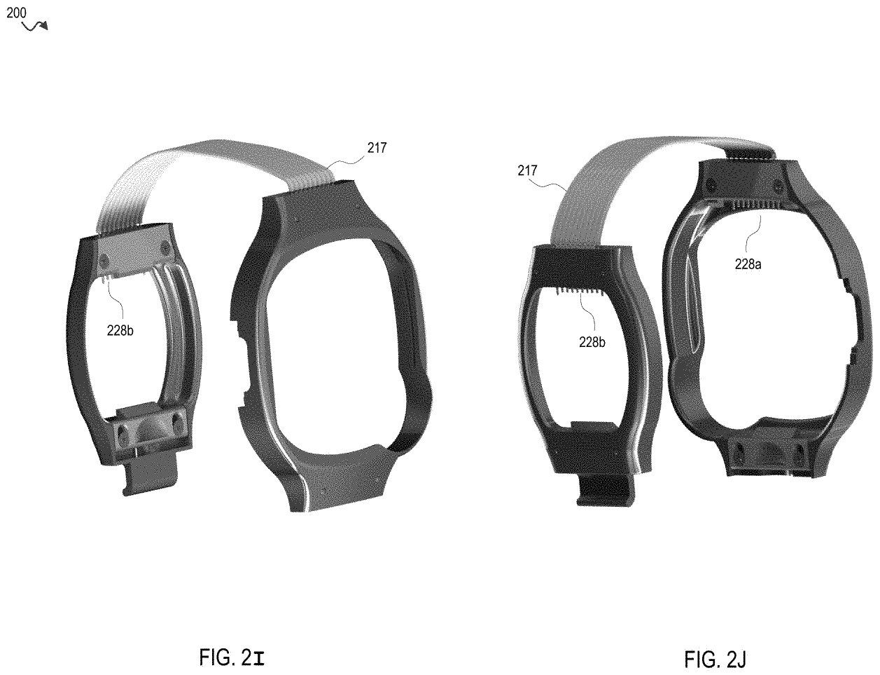

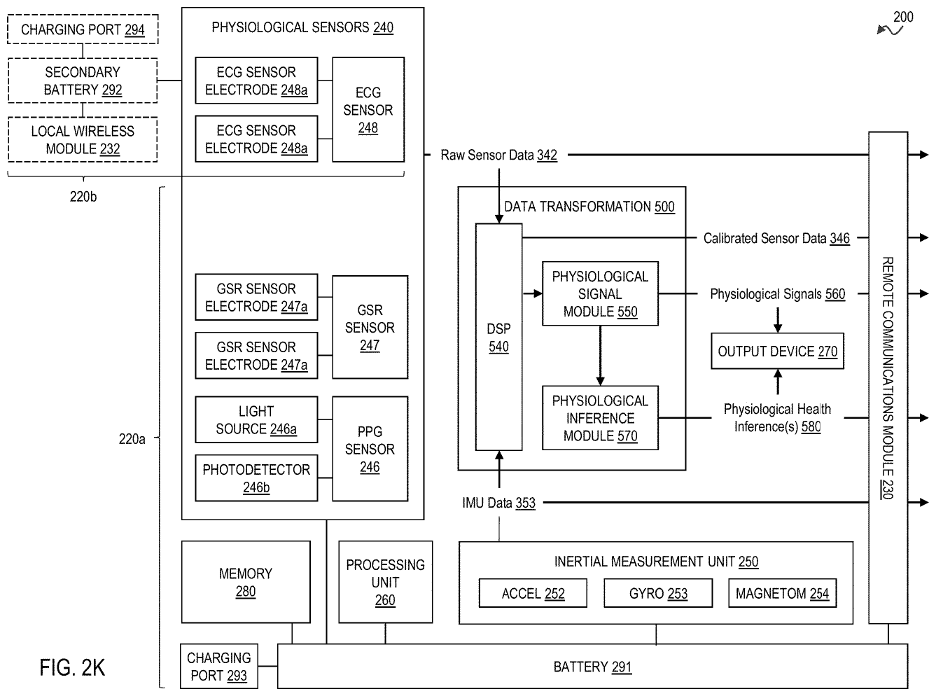

FIGS. 2A-2B are views of a wearable health monitoring device 200 according to an exemplary embodiment. As shown in FIGS. 2A-2B, the wearable health monitoring device 200 includes two sensor modules 220a and 220b connected to wristband segments 210a and 210b to form a wristband 210. The sensor module 220a includes an output device 270 (in this embodiment, a display).

In the embodiment of FIGS. 2A-2B, the sensor module 220a includes a PPG sensor 246 (having a light source 246a and a photodetector 246b) and a GSR sensor 247 (having GSR sensor electrodes 247a and 247b) and the sensor module 220b includes an ECG sensor 248 (having ECG sensor electrodes 248a and 248b shown in FIG. 2K and described below). However, in other embodiments, the wearable health monitoring device 200 may include any of a number of different physiological and other sensors. In fact, as described below, either or both of the sensor modules 220a and 220b may be removable and replaceable, enabling the wearable health monitoring device 200 to include different sensors as needed for specific applications. For example, for an individual or organization in the mining industry, the wearable health monitoring device 200 may include a sensor module that includes a number of gas sensors.

In the embodiment of FIGS. 2A-2B, the sensor module 220a includes a charging port 293 for charging a battery 291 (shown in FIG. 2K and described below) that provides power to the sensor module 220a and the sensor module 220b via wiring 217 (e.g., flex circuitry) in the wristband 210. However, other embodiments may not include wiring 217. Instead, in those embodiments, the sensor module 220b may wirelessly communicates with the sensor module 220a via a direct, short range communication protocol (e.g., Zigbee, Bluetooth, etc.) and may include a battery and a charging port for providing power to the battery (as described below with reference to FIG. 2K).

FIGS. 2C-2D are views of the sensor modules 220a and 220b (removed from the wristband segments 210a and 210b) according to an exemplary embodiment. FIGS. 2E-2F are views of the sensor modules 220a and 220b and wiring 217 (removed from the wristband segments 210a and 210b) according to an exemplary embodiment. FIGS. 2G-2H are views of a sensor board 226a of the sensor module 220a and a sensor board 226b of the sensor module 220b according to an exemplary embodiment. In the embodiment of FIG. 2G, the sensor module 220a also includes an inertial measurement unit 250 and a communications module 230.

FIGS. 2I-2J are views of a sensor module connection port 228a for the sensor module 220a and a sensor module connection port 228b for the sensor module 220b. As shown in FIGS. 2I and 2J, the 200 enables sensor modules to be removed, reconnected, and/or replaced with a different sensor module having different physiological or other sensors.

FIG. 2K is a block diagram of the wearable health monitoring device 200 according to exemplary embodiments.

As shown in FIG. 2K, the wearable health monitoring device 200 includes two sensor modules 220a and 220b, each with one or more sensors 222a and 222b. The sensors 222a and 222b include physiological sensors 240. The wearable health monitoring device 200 also includes a remote communications module 230, an inertial measurement unit 250, a hardware computer processing unit 260, output device(s) 270, memory 280, a battery 291, a charging port 293, and data transformation modules 500.

In the embodiment of FIG. 2K, the remote communications module 230 enables the wearable health monitoring device 200 to output data for transmittal to a local computing device 140. The remote communications module 230 may include, for example, a module for short range, direct, wireless communication (e.g., Bluetooth, Zigbee, etc.) and/or a module for communicating via a local area network (e.g., WiFi). In other embodiments, the remote communications module 230 may enable the wearable health monitoring device 200 to bidirectionally communicate with the server 160 via the one or more networks 150.

The output device 270 may include a display (e.g., as shown in FIGS. 2A-2H), a speaker, a haptic feedback device, etc. The memory 280 may include any non-transitory computer readable storage media (e.g., a hard drive, flash memory, etc.). The processing unit 260 may include any hardware computing device suitably programmed to perform the functions described herein (e.g., a central processing unit executing instructions stored in the memory 280, a state machine, a field programmable array, etc.)

The battery 291 provides power to the sensor module 220a. In some embodiments, the battery 291 also provides power to the sensor module 220b via the wire 217 described above. In those embodiments, the sensor module 220b transfers data (e.g., output by the ECG sensor 248) to the sensor module 220a via the wire 217. In other embodiments, however, the sensor module 220b wirelessly communicates with the sensor module 220a via a direct, short range communication protocol (e.g., Zigbee, Bluetooth, etc.). In those embodiments, the sensor module 220b may also include a local wireless module 232 for sending data to the sensor module 220a. Additionally, in embodiments where power is not transmitted through the wiring 217, the sensor module 220b may include a secondary battery 292 and a charging port 294 for providing power to the secondary battery 292.

The charging port 293 (and the charging port 294) may be hardware ports for receiving electrical power (e.g., a universal serial bus port, an inductive charging port, etc.)

The physiological sensors 240 may include any device capable of sensing data indicative of a physiological or biochemical condition of the wearer. In the embodiment of FIG. 2K, the physiological sensors 240 include a PPG sensor 246 having a light source 246a and a photodetector 246b, a GSR sensor 247 having GSR sensor electrodes 247a and 247b, and an ECG sensor 248 having ECG sensor electrodes 248a and 248b. The PPG sensor 246 may be any device capable of obtaining (e.g., optically) a plethysmogram that can be used to detect blood volume changes in the microvascular bed of tissue. The GSR sensor 247 may be any device capable of sensing the electrical conductance of the skin (i.e., the galvanic skin response). The ECG sensor 248 may be any device capable of sensing electrical signals generated by the beating heart of the wearer.

The inertial measurement unit 250 may be any device capable of measuring and reporting the specific force and angular rate of the wearable health monitoring device 200. The inertial measurement unit 250 may also measure and report the orientation of the wearable health monitoring device 200. In the embodiment of FIG. 2K, the inertial measurement unit 250 includes an accelerometer 252 (e.g., a 3-axis accelerometer), a gyroscope 253, and a magnetometer 254.

The inertial measurement unit 250 outputs IMU data 353 indicative of the movement of the wearable health monitoring device 200. The physiological sensors 240 output raw sensor data 342 indicative of a physiological or biochemical condition of the user. The remote communications module 230 outputs the IMU data 353 and the raw sensor data 342 for transmittal to the server 160 (e.g., via a local computing device 140).

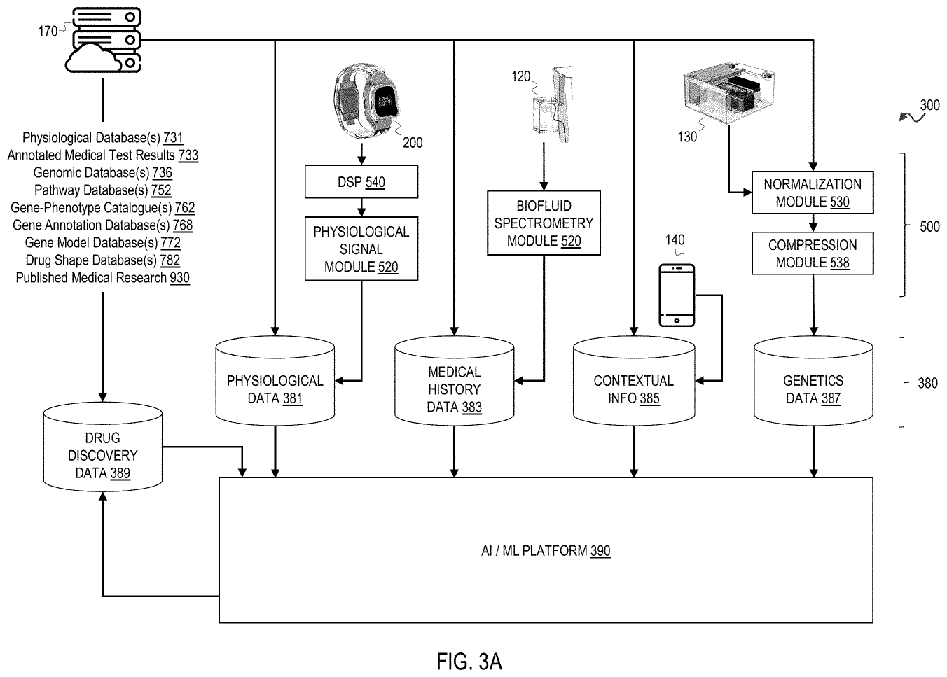

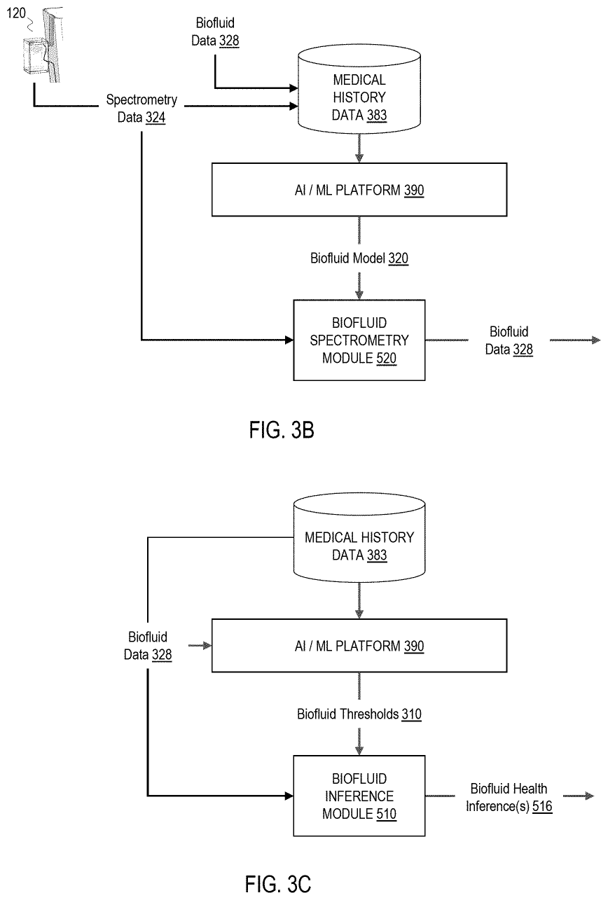

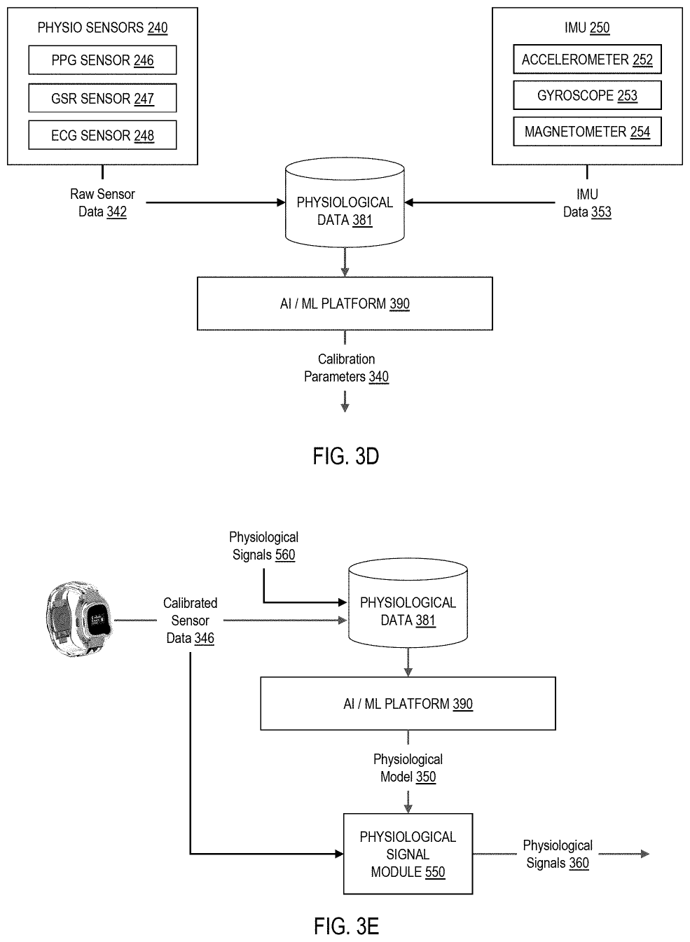

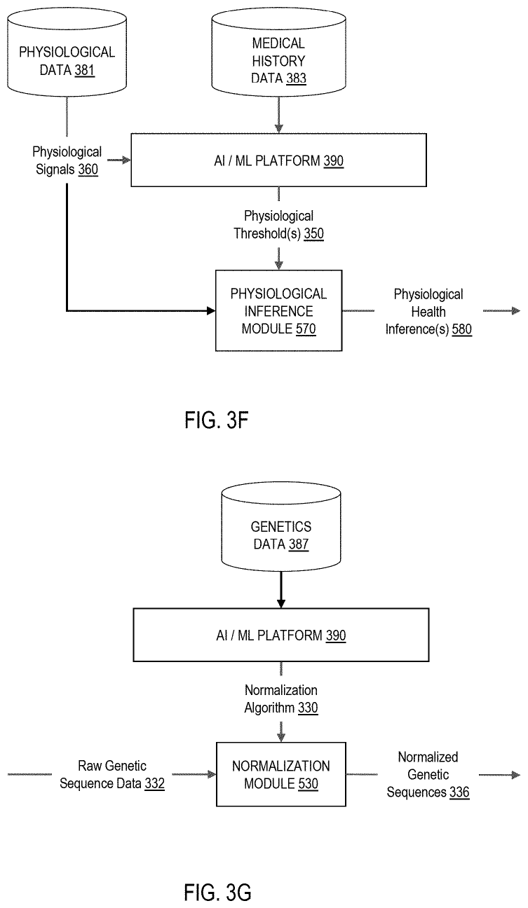

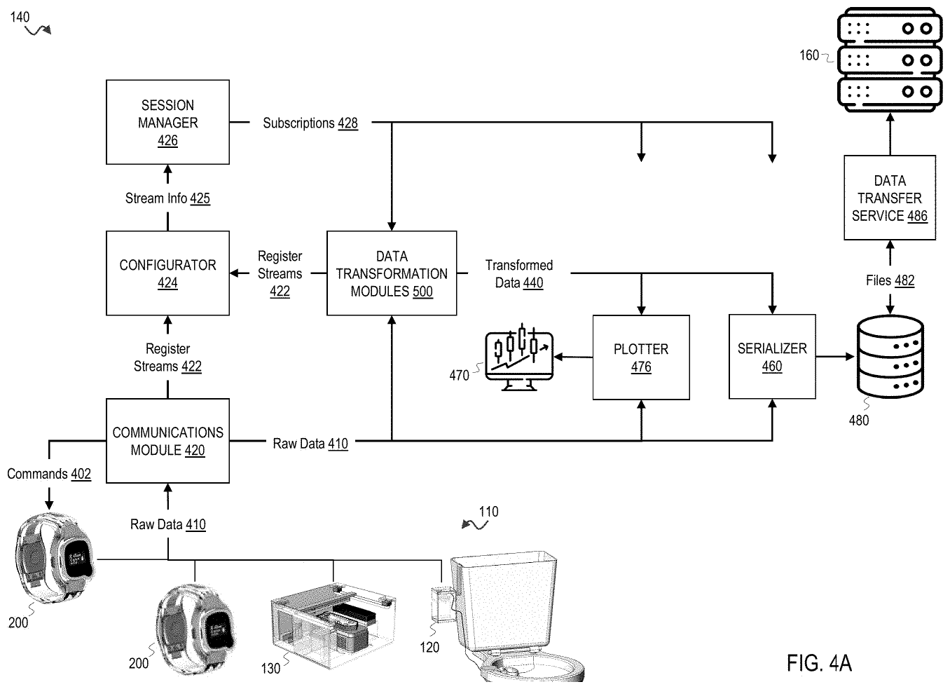

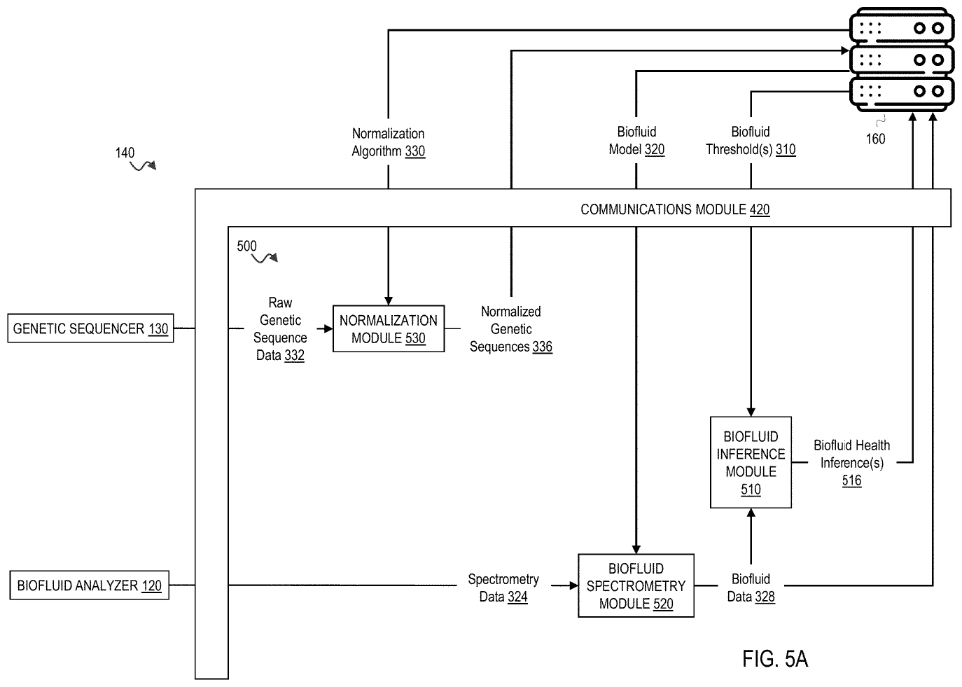

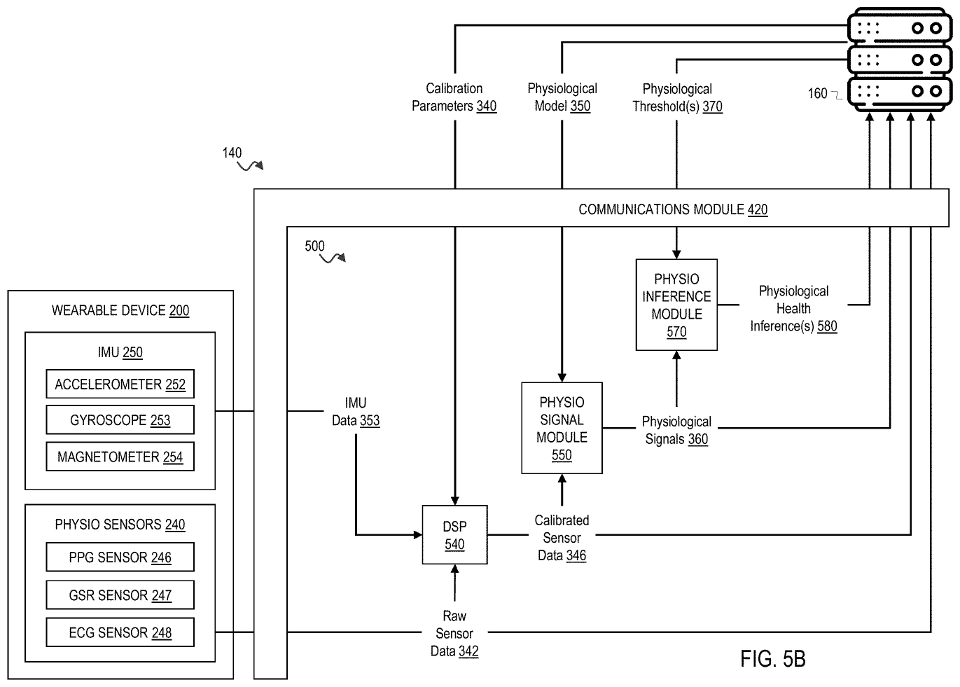

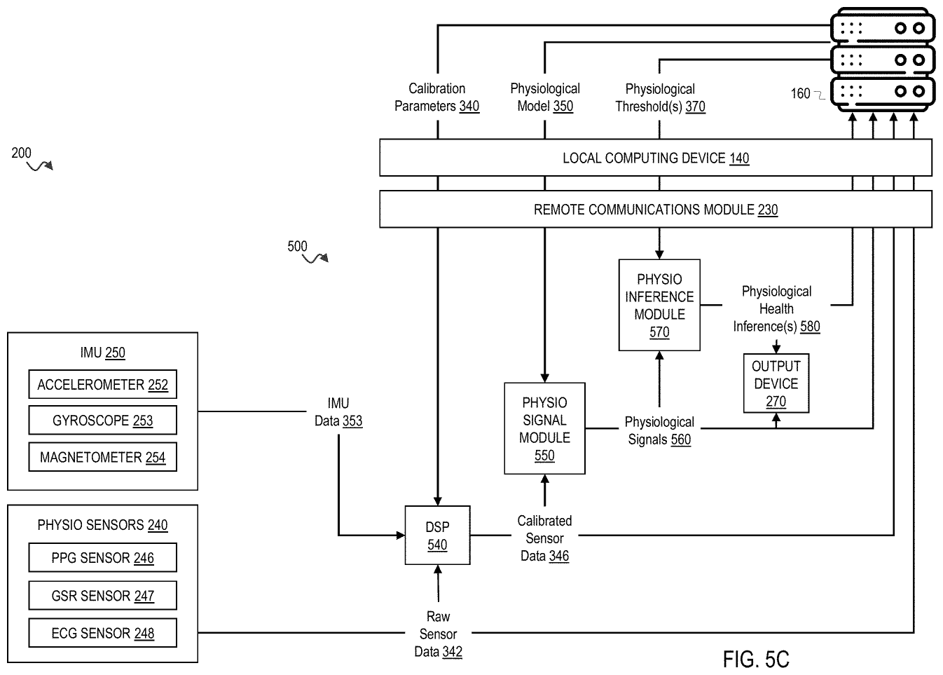

In some embodiments, the wearable health monitoring device 200 also includes data transformation modules 500, which are described in detail below with reference to FIGS. 3D-3F and 5B-5C. In the embodiment of FIG. 2K, for example, the wearable health monitoring device 200 includes a digital signal processing module 540 that performs digital signal processing on the raw sensor data 342 (e.g., to remove motion artifacts and/or noise) and generates calibrated sensor data 346, a physiological signal module 540 that identifies physiological signals 560 based on the calibrated sensor data 346, and a physiological inference module 540 that makes physiological health inferences 580 based on those physiological signals 580. The remote communications module 230 outputs the calibrated sensor data 346, the physiological signals 560, and any physiological health inferences 580 for transmittal to the server 160 (e.g., via a local computing device 140). In some embodiments, the physiological signals 580 may also be output to the user via an output device 270 (e.g., displayed to the user via a display). Physiological health inferences 580 may also be output to the user via an output device 270. For example, a visual, audible, and/or tactile alert may output to the user via display, a speaker, and/or a haptic feedback device.

### AI-Enabled Health Ecosystem 300

FIG. 3 is a diagram of the AI-enabled health ecosystem 300 according to exemplary embodiments.

As shown in FIG. 3A, the AI-enabled health ecosystem 300 stores health data 380, including physiological data 381 (e.g., in the physiological database 181 described above), medical history data 383 (e.g., in the medical history database 183), contextual information 385 (e.g., in the contextual information database 185), genetics data 387 (e.g., in the genetics database 187), and drug discovery data 389 (in the drug discovery database 189).

The physiological data 381 may include any information indicative of the physiological condition of humans. The physiological data 381 may be received from the wearable health monitoring device 200 and/or third-party computer systems 170 (e.g., electronic medical records systems, databases with physiological data collected from wearable health monitoring devices, etc.).

The medical history data 383 may include any information indicative of the medical history of humans. The medical history data 793 may be received from the biofluid analyzer 120 and/or third-party computer systems 170 (e.g., electronic medical records systems).

The contextual information 385 may include demographic information, medications taken that day, food journal containing diet and nutrients consumed, sleep hygiene/recovery status, stress management activities during the day, daily activity list, emotional state throughout the day, weather conditions, environmental and air pollution daily statistics, education status, financial status, childhood neighborhood, current neighborhood, access to nutritionally dense food, current and past socioeconomic status, social media use, urban/rural locations, etc. The contextual information 385 may be received from third-party computer systems 170 (e.g., electronic medical records systems). Additionally, the contextual information 385 may be input via local processing devices 140, for example by answering survey questions prompted by a software application (a web application, a smartphone application, a desktop application, etc.) the AI-enabled health ecosystem 300.

The genetics data 387 may include any information indicative of the nucleotide sequences of humans. For at least some of the individuals having medical data 380 in the dataset, the genetics data 387 includes the quantity of RNA for each of a number of genes in one or more biological samples. The genetics data 387 may be received from the genetic sequencer 130 and/or third-party computer systems 170 (e.g., electronic medical records systems).

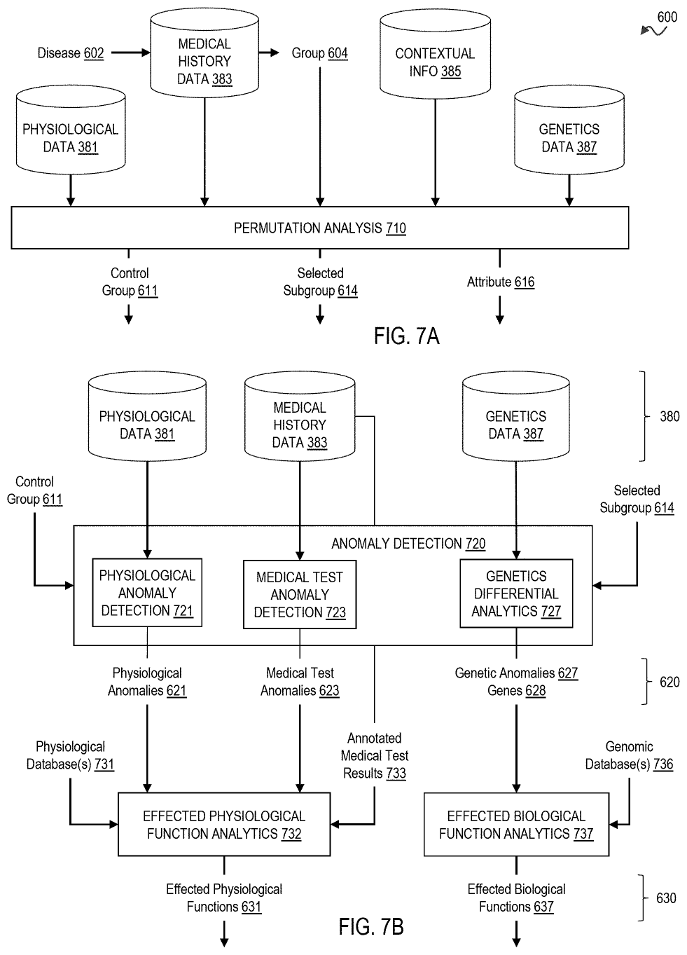

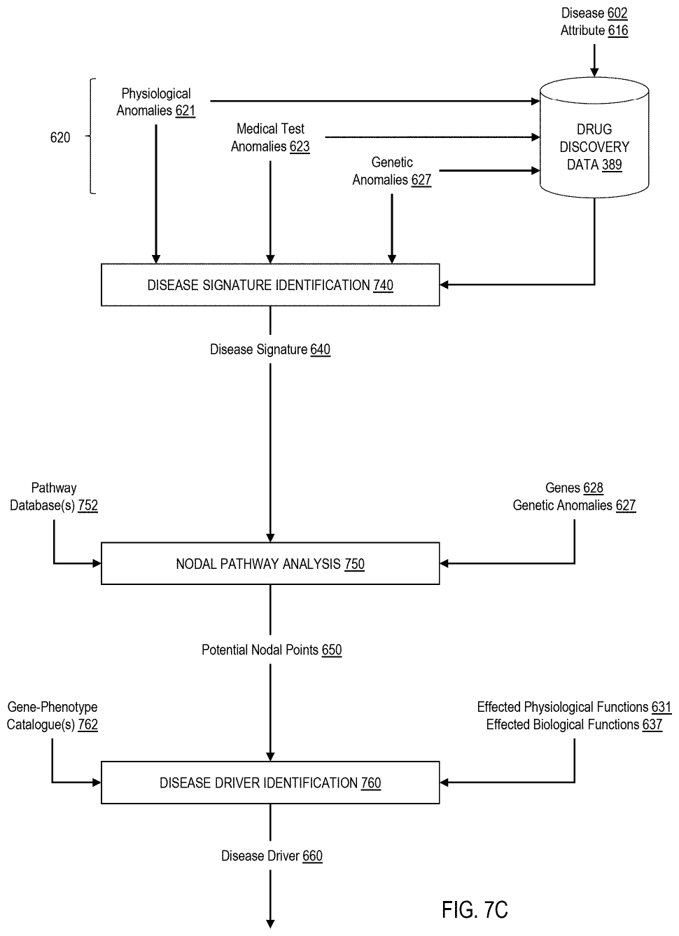

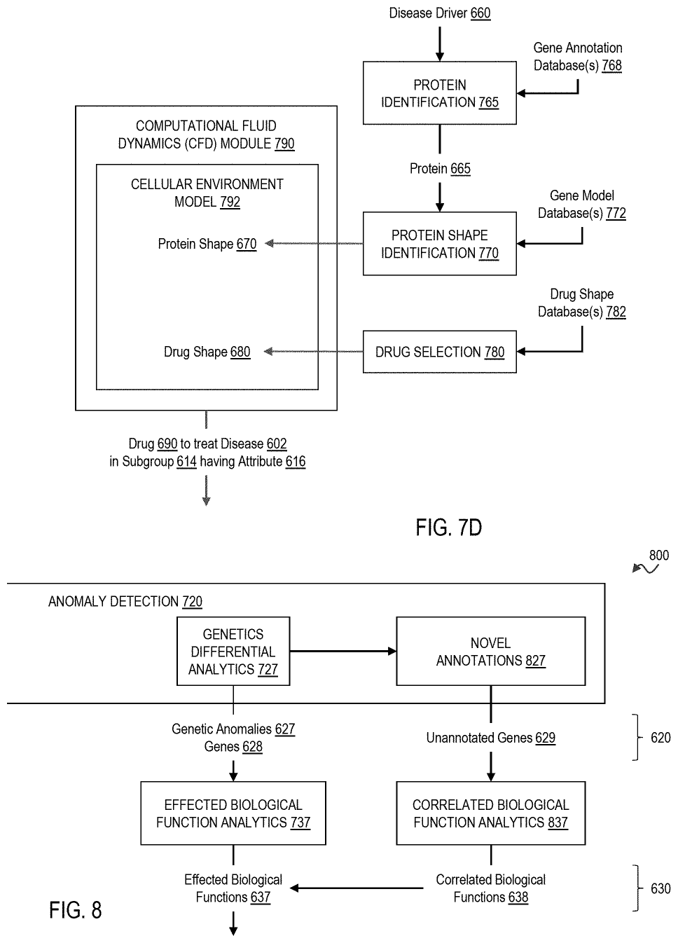

As described below with reference to FIGS. 7-9, the AI-enabled health ecosystem 300 stores drug discovery data 389, including information from one or more physiological databases 731 (e.g., The Physiome Project, PhysioNet, etc.), annotated medical test results (received from third-party computer systems 170 and/or stored as part of the medical history data 383), genomic databases 726 (e.g., European Genome-Phenome Archive, National Center for Biotechnology Gene Expression Omnibus (NCBI GEO), etc.), pathway database(s) 752 (e.g., Reactome, WikiPathways, MetaCyc, the Kyoto Encyclopedia of Genes and Genomes (KEGG), etc.), gene-phenotype catalogues 762 (e.g., the Online Mendelian Inheritance in Man (OMIM), etc.), gene annotation databases 768 (e.g., the Gene Ontology (GO), Database for Annotation, Visualization and Integrated Discovery (DAVID) etc.), gene model databases 772 (e.g., Protein Data Bank (PDB), etc.), drug shape databases 782 (e.g., LigandBook, ChEMBL, DrugBank, etc.), and published medical research 930.

The AI-enabled health ecosystem 300 also includes an artificial intelligence/machine learning platform 390 that uses the stored health data 380 to develop algorithms for a number of data transformation modules 500, for example a digital signal processing module 540 and a physiological signal module 520 (briefly mentioned above with reference to FIG. 3I) used to process raw sensor data 342 from wearable health monitoring devices 200, a biofluid spectrometry module 520 used to process spectrometry data captured by the biofluid analyzer 120, and a normalization module 530 and a compression module 538 used to normalize and compress genetic sequence data captured by the genetic sequencer 130.

FIG. 3B is a diagram illustrating a process for generating a biofluid model 320, executed by the biofluid spectrometry module 520, used to identify biofluid data 328 based on spectrometry data 324 output by the biofluid analyzer 120. As shown in FIG. 3B, the artificial intelligence/machine learning platform 390 is trained on a dataset (stored, for example, in the medical test data 383) that includes spectrometry data 324 captured by the biofluid analyzer 120 and biofluid data 328 captured by other, more precise biofluid analyzers (for example, in a clinical trial where biofluid samples are provided to both the biofluid analyzer 120 and one or more high precision biofluid analyzers such as a Kaglia Biosciences Biofluid Analyzer). The artificial intelligence/machine learning platform 390 then uses artificial intelligence and/or machine learning, trained on the dataset of spectrometry data 324 and biofluid data 328, to identify correlations between the spectrometry data 324 and the biofluid data 328 and generate a biofluid model 320 that generates biofluid data 328 based on spectrometry data 324.

FIG. 3C is a diagram illustrating a process for identifying biofluid thresholds 310 used by a biofluid inference module 510 to make biofluid health inferences 310. As described above, the AI-enabled health ecosystem 300 stores medical test data 383 that includes both biofluid data 328 and the medical history of those who provided that biofluid. Accordingly, the artificial intelligence/machine learning platform 390 is trained on that dataset to identify correlations between biofluid data 328 and medical conditions and identifies biofluid thresholds 310 indicative of medical conditions. For example, sugar in urine is indicative of diabetes issues. Those biofluid thresholds 310 are provided to the biofluid inference module 510, which outputs a biofluid health inference 516 in response to a determination that biofluid data 328 meets or exceeds one the provided biofluid thresholds 310.

FIG. 3D is a diagram illustrating a process for identifying calibration parameters 340 used by the digital signal processing module 540 to remove motion artifacts and/or noise from the raw sensor data 342 output by the physiological sensors 240 of the wearable health monitoring device 200.

As briefly mentioned above with reference to FIG. 3I, the raw sensor data 342 output by the physiological sensors 240 of the wearable health monitoring device 200 may be corrupted by motion artifacts due to motion of the wearable health monitoring device 200. To remove those motion artifacts/noise, the digital signal processing module 540 may use any number of statistical signal processing techniques, including adaptive filters, static highpass or bandpass filtering, etc. In some embodiments, an adaptive filter may be utilized that incorporates an acceleration measurement as a reference signal. Accordingly, in those embodiments, the wearable health monitoring device 200 includes an inertial measurement unit 250 that includes a 3-axis accelerometer 252. In some of those embodiments, the inertial measurement unit 250 may further include a 3-axis gyroscope 253 and a 3-axis magnetometer 254, etc., which may be utilized to perform sensor fusion in order to estimate the gravity vector measured by the accelerometer 252. The digital signal processor 250 may then remove motion artifacts from the raw sensor data 342 utilizing, for example, an adaptive filter using the reduced variance accelerometer measurements.

FIG. 3E is a diagram illustrating a process for generating a physiological model 350, executed by the physiological signal module 550, used to identify physiological signals 360 based on calibrated sensor data 346 output by the physiological sensors 240 of the wearable health monitoring device 200. As shown in FIG. 3E, the artificial intelligence/machine learning platform 390 is trained on a dataset (stored, for example, in the physiological data 381) that includes calibrated sensor data 346 output by the wearable health monitoring device 200 and the physiological signals 560 captured by other, more precise physiological sensors (for example, during a clinical trial where participants wear the wearable health monitoring device 200 while their physiological signals 560 are also captured by hospital-grade monitors such as the Empatica E4 wristband). The artificial intelligence/machine learning platform 390 then uses artificial intelligence and/or machine learning, trained on the dataset of the calibrated sensor data 346 and the physiological signals 560, to identify correlations between the calibrated sensor data 346 and the physiological signals 560 and generate a physiological model 350 that generates physiological signals 560 based on calibrated sensor data 346.

FIG. 3F is a diagram illustrating a process for identifying physiological thresholds 370 used by the physiological inference module 570 to make physiological health inferences 580. As described above, the AI-enabled health ecosystem 300 stores both patient medical histories (the medical history data 383) and the physiological signals 360 of some of those patients (the physiological data). Accordingly, the artificial intelligence/machine learning platform 390 is trained on that dataset to identify correlations between physiological signals 360 and medical conditions and, for each physiological signal 360, identifies one or more physiological thresholds 370 indicative of a medical condition. For example, a blood pressure reading of 140/90 mm Hg may be indicative of hypertension. Those physiological thresholds 370 are provided to the physiological inference module 570, which outputs a physiological health inference 580 in response to a determination that a physiological signal 360 meets or exceeds a physiological threshold 350 indicative of a medical condition.

FIG. 3G is a diagram of the process for generating a normalization algorithm 330 used by the normalization module to normalize raw genetic sequence data 332 and generate normalized genetic sequences 336.

### Local Computing Device 140

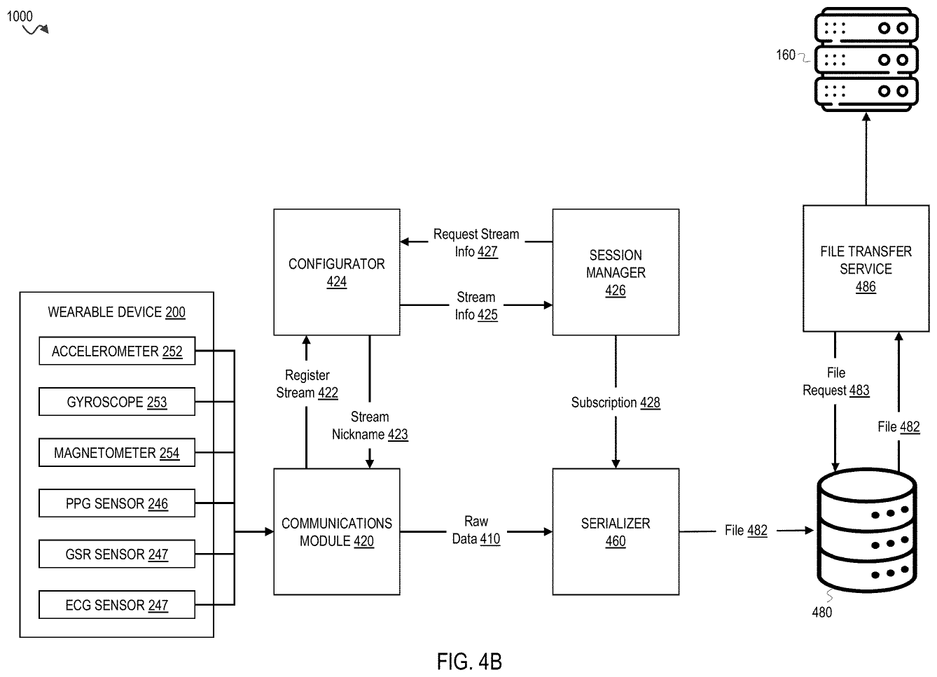

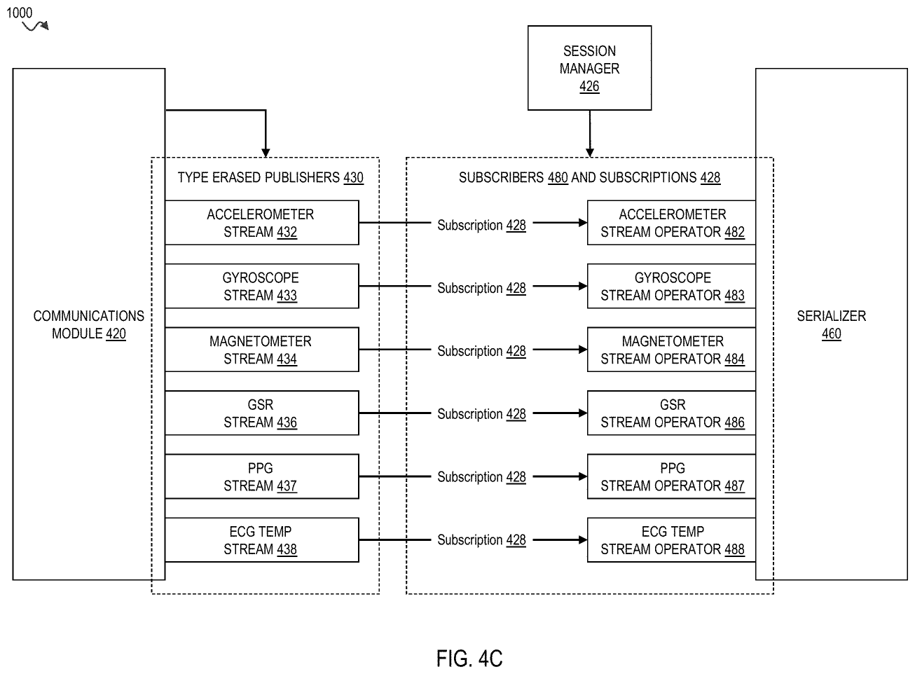

FIG. 4A is a block diagram of a local computing device 140 according to exemplary embodiments.

In the embodiments of FIG. 4A, the local computing device 140 includes a communications module 420, a configurator 424, a session manager 426, a data transformer 430, a serializer 460, local storage 480, and a data transfer service 486. In some embodiments, the local computing device 140 also includes a plotter 476 and a user interface 470.

The communications module 420 receives raw data 410 (e.g., in binary format) from one or more data acquisition devices 110. The raw data 410 may include, for example, raw sensor data 342 output by the physiological sensors 240 of the wearable health monitoring device 200, raw genetic sequence data 332 output by the genetic sequencer 130, spectrometry data output by the biofluid analyzer 120, etc. Because some data acquisition devices 110 (such as the wearable health monitoring device 200) may include multiple sensors, the raw data 410 may include data from multiple sensors. The communications module 420 may also output commands 402 to one or more of the data acquisition devices 110 (e.g., using a commands application programming interface (API)).

The communications module 420 parses the raw data 410 and publishes the raw data 410 as data streams. Modules that produce one or more data streams (e.g., the communications module 420, the data transformer(s) 430, the serializer 460, and the plotter 470) are referred to as “stream producers.” Conversely, modules that consume one or more data streams (e.g., the data transformer(s) 430, the serializer 460, the plotter 470) are referred to herein as “stream consumers.” The produced streams are registered with the configurator module 420 (register streams 422), which acts as the middleware between stream producers and stream consumers. The session manager 426 manages the different sessions in the application, depending on what is needed for a particular use case, by outputting subscriptions 428 to the stream consumers.

Data transformation module(s) 500 process the raw data 410 to generate transformed data 440. As described above with reference to FIGS. 3B-3G and below with reference to FIGS. 5A-5C, the data transformation module(s) 500 may perform digital signal processing (e.g., to remove motion artifacts from sensor data, remove noise from PPG data, etc.), batch normalize genetic sequencing data, detect anomalies in biofluid data or physiological signals indicative of changing health or a disease state, etc.

The serializer module 460 serializes the raw data 410 and the transformed data 440 into a supported serialization format (e.g., JavaScript object notation (JSON), ProtoBufs and FlatBuffer) and stores the serialized data as files in the local storage 480. The data transfer service 486 uploads the files from the local storage 480, either in batches or in near real time (i.e., a streaming mode). The data transfer service 486 may be, for example, a state machine. In embodiments that include a user interface 470, the plotter module 476 configures and plots the transformed data 440 for display via the user interface 470.

### Type Erasure

A strongly typed programming language is one in which variables are bound to specific data types. Strongly typed programming languages enable better performance. However, in applications programmed using a strongly typed programming language, data types in expressions that do not match up as expected result in type errors. To improve performance, the software application may utilize a strongly typed programming language (e.g., Swift). However, the raw data 410 received from the data acquisition devices 110 (e.g., physiological data received from the wearable health monitoring device 200) may be heterogeneous data with different bit depths. Therefore, to store that heterogeneous raw data 510 as variables and avoid the type errors generated by strongly typed programming languages, the application may perform a type erasure process on the received physiological data.

FIGS. 4B and 4C are block diagrams of a type erasure process 400 according to an exemplary embodiment. As described above, the wearable health monitoring device 200 may include an accelerometer 252, a gyroscope 253, a magnetometer 254, a PPG sensor 246, a GSR sensor 247, and an ECG sensor 247. The raw data 410 (e.g., the raw sensor data 342 and IMU data 353, etc.) output by the wearable health monitoring device 200 is received by the communications module 420 of the local processing device 140, where the raw data 410 is serialized by the serializer 460 and stored as files 482 in the local storage 480 and transferred to the server 160 by the file transfer service 486.

As shown in FIG. 4B, publishers created by the communications module 420 are registered with the configurator 424 as data streams (register stream 422) that are tagged with a stream nickname 423 and one or more flags indicating intended use. For example, a data stream can be tagged with a flag “isSerializable” indicating that this stream may be serialized, if the session manager 426 should deem it necessary. The session manager 426 requests the configurator 424 for the data streams that are serializable (request stream info 427) and creates the necessary stream consumers and the subscriptions 428 to hook up the streamers with the stream consumers. The serializer 460 serializes and persists the raw data 410 in the local storage 480 as files 482 in a serialization format (e.g., JSON, ProtoBufs and FlatBuffer). The file transfer service 486 (e.g., a state machine) monitors the local storage 480 for new files 482 that need to be uploaded to the server 160, requests those files (file request 483), and uploads the files 482. The file transfer service 486 may take into account the network connectivity to the server 160 and try to leverage the hardware capabilities of the local processing device 140. For example, in an iOS device, the file transfer service 486 takes advantage of the iOS scheduler to decide when it is a good time to schedule the transfer (taking into account the current battery life, the charging state, the expected future use of the device, etc.)

As shown in FIG. 4C, six data streams may be created (one for each of the sensors of the wearable health monitoring device 200) by the communications module 420 and consumed by the serializer module 460. In the embodiment of FIG. 4C, a type erased publisher 430 is dynamically created for each of the six data streams, for example by the communications module 420. In the embodiment of FIG. 4C, the type erased publishers 430 include an accelerometer stream 432, a gyroscope stream 433, a magnetometer stream 434, a GSR stream 436, a PPG stream 437, and an ECG stream 438. The data streams created by these publishers are type erased and consumed by subscribers 460 after a subscription 428 is made. In the embodiment of FIG. 4C, the subscribers 480 include an accelerometer stream operator 482, a gyroscope stream operator 483, a magnetometer stream operator 484, a GSR stream operator 486, a PPG stream operator 487, and an ECG stream operator 488. The subscribers 480 and subscriptions 428 may be dynamically created by the stream consumer (in this example, the serializer module 460). Alternatively, the subscribers and subscriptions 428 may be dynamically created by some orchestrator system, for example the session manager 426.

### Data Transformation Modules 500

FIGS. 5A-5C are block diagrams of data transformation modules 500 according to exemplary embodiments. In the embodiment of FIG. 5A, the local processing device 140 includes the normalization module 530, the biofluid spectrometry module 520, and the biofluid inference module 510. The normalization module 530 normalizes raw genetic sequence data 332 (received, for example, from the genetic sequencer 130 via the communications module 420) and outputs normalized genetic sequences 336. To normalize the raw genetic sequence data 332, the local processing device 140 receives the normalization algorithm 330 (generated by the artificial intelligence/machine learning platform 390 as described above) from the server 160 via the communications module 420.

The biofluid spectrometry module 520 receives spectrometry data 324 (received, for example, from the biofluid analyzer 120 via the communications module 420) and outputs biofluid data 328. To generate the biofluid data 328 based on the spectrometry data 324, the local processing device 140 receives the biofluid model 320 (generated by the artificial intelligence/machine learning platform 390 as described above) from the server 160 via the communications module 420.

The biofluid inference module 510 is used to make biofluid health inferences 516, for example by detecting anomalies in the biofluid data 328. To make biofluid health inferences 516 based on the biofluid data 328, the local processing device 140 receives biofluid thresholds 310 (generated by the artificial intelligence/machine learning platform 390 as described above) from the server 160 via the communications module 420.

In the embodiment of FIG. 5B, the local processing device 140 includes the digital signal processing module 540, the physiological signal module 550, and the physiological inference module 570. The digital signal processing module 540 performs digital signal processing to remove motion artifacts and/or noise from raw sensor data 342 (received, for example, from the wearable health monitoring device 200 via the communications module 420). To do so, the digital signal processing module 540 receives calibration parameters 340 (generated by the artificial intelligence/machine learning platform 390 as described above) from the server 160 via the communications module 420.

The physiological signal module 550 identifies physiological signals 360 based on the calibrated sensor data 346. To generate the physiological signals 360 based on the calibrated sensor data 346, the local processing device 140 receives the physiological model 350 (generated by the artificial intelligence/machine learning platform 390 as described above) from the server 160 via the communications module 420.

The physiological inference module 570 is used to make physiological health inferences 580, for example by detecting anomalies in one or more of the physiological signals 360. To make physiological health inferences 580 based on the physiological signals 360, the local processing device 140 receives physiological thresholds 370 (generated by the artificial intelligence/machine learning platform 390 as described above) from the server 160 via the communications module 420.

In the embodiment of FIG. 5C, the wearable health monitoring device 200 includes the digital signal processing module 540, the physiological signal module 550, and the physiological inference module 570 and receives the calibration parameters 340, the physiological model 350, and the physiological thresholds 370 from the server 160 via the remote communications module 230 and the local computing device 140.

### Personalized, Genetics-Based Drug Discovery

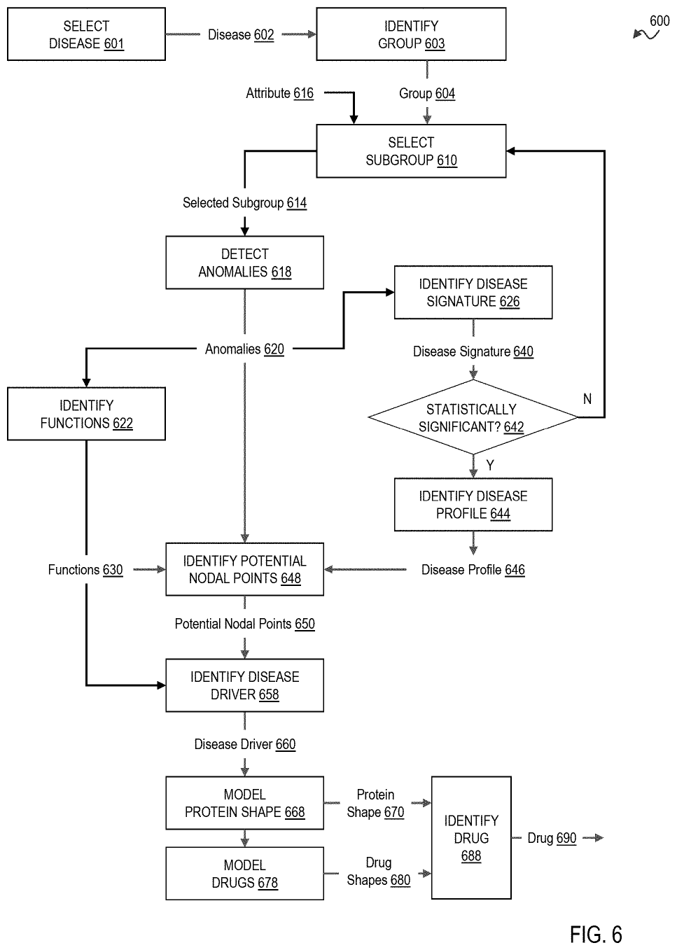

FIG. 6 is a flowchart illustrating a personalized drug discovery process 600, which is described in greater specificity and detail in FIGS. 7A through 7D, according to exemplary embodiments. The personalized drug discovery process 600 may be performed, for example, by the server 160 of the AI-enabled health ecosystem 300 described above.

As shown in FIG. 6, the personalized drug discovery process 600 identifies a drug 690 to treat a disease 602 in a subgroup 614 of individuals having a common attribute 616 (e.g., members of a specific demographic group, having another medical condition in addition to the disease 602, etc.)

A disease 602 is selected in step 601. A group 604 of individuals having the disease 602 is identified in step 603. A subgroup 614 of the group 604 having a common attribute 616 is selected in step 610. Anomalies 620 are detected in the medical data 380 of the selected subgroup 614 in step 618. The functions 630 effected by those anomalies 620 are identified in step 622.

The process 600 is recursive, with subgroups 614 having common attributes 616 being repeatedly selected until a subgroup 614 is identified with a disease signature 640 (i.e., the anomalies 620 prevalent in the selected subgroup 614 that are not prevalent the control group 611) that is statistically significant as compared to a control group 611. The disease signature 640 for the selected subgroup 614 is identified in step 626. If the disease signature is not statistically significant compared to the control group (Step 642: No), the process returns to step 610 and another subgroup 614 having a different attribute 616 is selected. If the disease signature 640 for the selected subgroup 614 is statistically significant (Step 642: Yes)

A disease profile 646 is identified in step 644. To do so, the anomalies 620 detected in the selected subgroup 614 are compared to the anomalies 620 previously detected for previously selected subgroups 614 having other attributes 616 in common.

Potential nodal points 650 are identified in step 648 based on the identified anomalies 620, the disease signature 640, and the disease profile 646. The disease driver 660 in step 658 based on the effected functions 630. If a protein coding gene is identified, then a drug 690 that binds to a protein made by the disease driver 660 is selected in step 688. To do so, the protein conformation 670 is modeled in step 668 and drug structure 680 are modeled in step 678. If a ncRNA is identified a different workflow will be used. The ncRNA itself could be made into a drug, or, by examining the regulatory pathways involved in the ncRNA life cycles, many of which are protein coding, can be identified as targets instead.

As described above, the personalized drug discovery process 600 can be performed (e.g., by the server 160) to identify the drug 690 having the most efficacy in treating the disease 602 for individuals having the attribute 616 (and the fewest side effects). If a satisfactory drug 690 to address the identified disease driver 660 cannot be identified—for example, if the disease driver 660 is difficult to address via pharmacology, a drug 690 that binds to the protein conformation 670 cannot be identified, identified drugs 690 are ineffective or have unsatisfactory side effects, etc.—another potential nodal point 650 may be selected as a potential disease driver 660 and steps 668, 678, and 688 can be repeated to identify a drug 690 to address the newly-selected disease driver 660.

FIGS. 7A through 7D are a flowchart illustrating a personalized, genetics-based drug discovery process 700 according to exemplary embodiments. The personalized, genetics-based drug discovery process 700 may be performed, for example, by the server 160 of the AI-enabled health ecosystem 300 described above. As one of ordinary skill in the art would recognize, some of the processing steps described below may be optional and may not be performed in each embodiment of the process 700. Additionally, the processing steps do not necessarily have to be performed in the order shown in FIGS. 7A-7D and described below.

The genetics-based process 700 described below is similar to the (more generic) drug discovery process 600 described above with reference to FIG. 6. However, as described below, the genetics-based process 700 leverages the AI-enabled health ecosystem 300—specifically, the combination of physiological data 381, medical history data 383, contextual information 385, and genetics data 387—to identify unique disease profiles in subgroups 614 that cannot be identified using conventional drug discovery processes. For example, the genetics-based drug discovery process 700 recently determined that a disease 602 caused a different genetic expression in women than men and, as a result, a drug 690 that had been tested in a clinical trial that mainly included men was not the most effective for treating the disease 602 in that subgroup 614 (i.e., women).

Additionally, combining genetics data 387 with physiological data 381 and medical history data 383 enables the genetics-based process 700 to better identify disease drivers 660 than traditional drug discovery processes and, by extension, to identify the drug 690 with the highest efficacy in treating that disease 602 in that subgroup 614.

As shown in FIG. 7A, the group 604 of individuals with the disease 602 is identified in the medical history data 383. A permutation analysis module 710 selects, from the group 604 with the disease 602, a subgroup 614 of individuals having a common attribute 616. As briefly mentioned above, the subgroup 614 may be members of a specific demographic group, individuals living in a specific location, individuals who eat a particular diet, individuals who live and/or work in particular environments (i.e. rural, industrial, high altitude, low altitude, high pollution, low pollution), individuals who are in a similar height/weight/age group, individuals who are of the same ethnic descent, individuals with physiological data 381 that includes one or more similar physiological signals 560, individuals with medical history data 383 that includes similar biofluid data 328 or another medical condition in addition to the disease 602 (related to or unrelated to the disease 602), individuals with similar genetic expressions and/or profiles, etc.

A control group 611 is also identified. The control group 611 may be, for example, healthy individuals, individuals without the disease 602, individuals with another disease (related or unrelated to the disease 602), etc.

As shown in FIG. 7B, the anomaly detection module 720 identifies anomalies 720 that are more common in the medical data 380 of the selected subgroup 614 than in the medical data 380 of the control group 611. Specifically, a physiological anomaly detection module 721 identifies physiological anomalies 621 in the physiological data 381, a medical test anomaly detection module 723 identifies medical test anomalies 623 in the medical history data 383, and a genetics differential analytics module 727 performs genetics differential analytics—for example, RNA sequencing (RNA-seq), variant calling, chromatin immunoprecipitation with massively parallel DNA sequencing (ChIP-seq), assay for transposase-accessible chromatin using sequencing (ATAC-seq), etc.—to identify genetic anomalies 627 (e.g., genes 628 that are producing more or less RNA than those genes produce in the control group 611.

The effected physiological function analytics module 732 searches the physiological database(s) 731 (e.g., The Physiome Project, PhysioNet, etc.) and suggests the effected physiological functions 631 of each physiological anomaly 731 identified in the physiological data 381 of the selected subgroup 614. For example, if the physiological anomalies 631 are ECG data with R-S intervals that are shorter than and R-peaks that are higher, an effected physiological function 631 is an arrythmia. The effected physiological function analytics 732 also searches annotated medical test results 733 (received from a third-party computer system 170 and/or stored in the medical history data 383 and suggests the effected physiological functions 631 of each medical test anomaly 626 identified in the medical history data 383 of the selected subgroup 614. For example, if the medical test anomaly 623 is high blood pressure, the effected physiological function 631 may be hypertension. Similarly, if the medical test anomaly 623 is a white dot on an X-ray of a lung, the effected physiological function 631 may be cancer (if the white dot is intense), tuberculosis (if the white dot is dispersed), etc.

The effected biological function analytics module 737 searches the genetic database(s) 736 (e.g., the Gene Ontology, KEGG, etc.) and identifies the effected biological functions 637 of each gene 628 with a genetic anomaly 627. For example, the genetic database(s) 736 may indicate that a group of genes 628 with genetic anomalies 627 are known to be related to cardiac conductance.

Like the drug discovery process 600 described above with reference to FIG. 6, the personalized, genetics-based drug discovery process 700 is recursive, with subgroups 614 having common attributes 616 being repeatedly selected until a subgroup 614 is identified with a statistically significant disease signature 640 is identified.

If the selected subgroup 614 demonstrates a statistically significant disease signature 640, a nodal pathway analysis module 750 identifies potential nodal points 650. The nodal pathway analysis unit 750 uses pathway database(s) 752 (e.g., Reactome, WikiPathways, MetaCyc, the Kyoto Encyclopedia of Genes and Genomes (KEGG), etc.) to identify the genetic pathway that includes the affected genes 628 having the identified genetic anomalies 627 and identifies the earliest genes 628 along that genetic pathway (the potential nodal points 650), which are likely to have caused the most genetic anomalies 627 along that genetic pathway.

A disease driver identification module 760 identifies, from among the potential nodal points 650, the most likely disease driver 660. The nodal pathway analysis unit 750 outputs the potential nodal points 650 as a list of nodal points 650 ranked by the likelihood that each is the disease driver 660. Additionally, the disease driver identification module 760 uses gene-phenotype catalogue(s) 762 (e.g., OMIM, etc.) to identify the genes 638 commonly associated with the effected physiological functions 631 and the effected biological functions 637 of the anomalies 620 identified in the medical data 380 of the subgroup 614. In some of the examples above, for instance, an effected physiological function 761 of the physiological anomalies 731 was an arrythmia and an effected biological function 767 of a group of genes 628 with a genetic anomaly 627 was cardiac conductance. Because abnormal cardiac conductance causes an arrythmia, in that instance the disease driver identification module 760 may identify one of those genes 628 as the disease driver 660 (i.e., the gene 628 along the nodal pathway most likely causing arrythmia).

Conventional drug discovery processes only examine either genetic pathways or physiological pathways. By contrast, because the AI-enabled health ecosystem 300 combines physiological data 381, medical history data 383, and genetics data 387, the drug discovery process 600 is able to identify both effected physiological functions 631 and effected biological functions 637 and use both physiological and biological information to identify the most likely disease driver 660 in the selected subgroup 614.

By identifying the most likely disease driver 660 of the disease 602 in individuals with the attribute 616, the drug discovery process 700 makes it possible to address the root cause of that disease (e.g., via a therapeutic, a lifestyle intervention, etc.) rather than addressing a symptom of that disease. For instance, while someone with hypertension may artificially lower their blood pressure through medication, that person has not identified the disease driver 660 causing that hypertension. By contrast, the drug discovery process 700 identifies the disease driver 660 for individuals with that attribute 616 and, as described below, identifies the drug 690 with the highest efficacy (and fewest side effects) in treating individuals with that disease 602 in that subgroup 614.

As shown in FIG. 7D, a protein identification module 765 searches gene annotation database(s) 768 (e.g., the Gene Ontology (GO), Database for Annotation, Visualization and Integrated Discovery (DAVID) etc.) and identifies a protein 665 made by the disease driver 660 (i.e., the gene 628 identified as the most likely causing the most number of genetic anomalies 627 along the genetic pathway). A protein shape identification module 770 searches gene model database(s) 772 (e.g., Protein Data Bank (PDB), etc.) and identifies a protein conformation 670 of a protein 665 produced by the disease driver 660.

A drug 690 to treat the disease 602 in the subgroup 614 having the attribute 616 is identified using computational fluid dynamics (CFD). A computational model of the human cellular environment (cellular environment model 792) is provided to a CFD module 790. The CFD module 790 models the protein conformation 670 in the cellular environment 792 and a drug selection module 780 searches drug shape database(s) 782 (e.g., LigandBook, ChEMBL, DrugBank, etc.) for a drug 690 with a drug shape 680 that binds to the protein 665 in the cellular environment 792.

As described above, the personalized, genetics-based drug discovery process 700 can be performed (e.g., by the server 160) to identify the drug 690 having the most efficacy in treating the disease 602 for individuals having the attribute 616 (and the fewest side effects). If a satisfactory drug 690 to address the identified disease driver 660 cannot be identified—for example, if the identified disease driver 660 is difficult to address via pharmacology, a drug 690 that binds to the protein conformation 670 cannot be identified, identified drugs 690 are ineffective or have unsatisfactory side effects, etc.—another potential nodal point 650 may be selected as a potential disease driver 660 by the disease driver identification module 760 and the process shown in FIG. 7D can be repeated to identify a drug 690 to address the newly-selected disease driver 660.

### Novel Annotations

In addition to genes 628 known to be associated with specific biological functions 637 (described above with reference to FIG. 7B), in some embodiments the AI-enabled health ecosystem 300 identifies genetic anomalies in genes that have yet to be annotated.

FIG. 8 is a diagram illustrating a process 800 for identifying anomalies 620 in unannotated genes 629 and potential functions 630 of those unannotated genes 629. A novel annotations module 827 performs genetics differential analytics (e.g., RNA-seq, ChIP-seq, ATAC-seq., etc.) to identify genetic anomalies 627 in unannotated genes 629. In those instances, the novel annotations module 827 may annotate those genes 629 (for example, as being potentially related to the effected physiological functions 631 identified by analyzing the physiological data 381 and medical history data 383 of the subgroup 614). Additionally, a correlated biological function analytics module 837 may identify potential effected biological functions 637 (correlated biological functions 683) by identifying functions associated with genes in other animals that are thought to be correlated with the unannotated genes 629.

### Modeling Human Conditions in Disease

As described above with reference to FIG. 7D, a computational model of the cellular environment (cellular environment model 792) is provided to the computation fluid dynamics module 790. However, conventional modeling of protein-drug binding occurs in a cellular environment model 792 approximating salt in a water-like environment with a simulated pH of ˜7. While that conventional cellular environment model 792 may be easy to simulate, the conventional cellular environment model 792 does not reflect the actual physiological environment of a disease702, which often involves an imbalance of electrolytes in both tissue and fluid.

Accordingly, in some embodiments, to more accurately simulate high-efficacy protein-drug modeling, the computational fluid dynamics module 790 models the binding of drugs 890 and proteins 665 in environments more closely reflecting the electrical charges and conditions of the diseased environment.

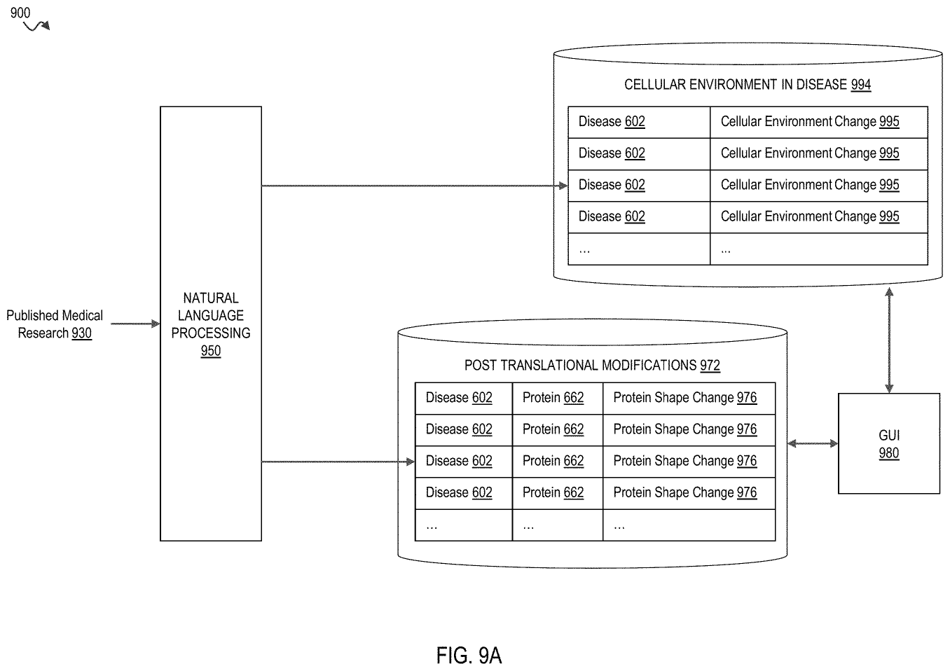

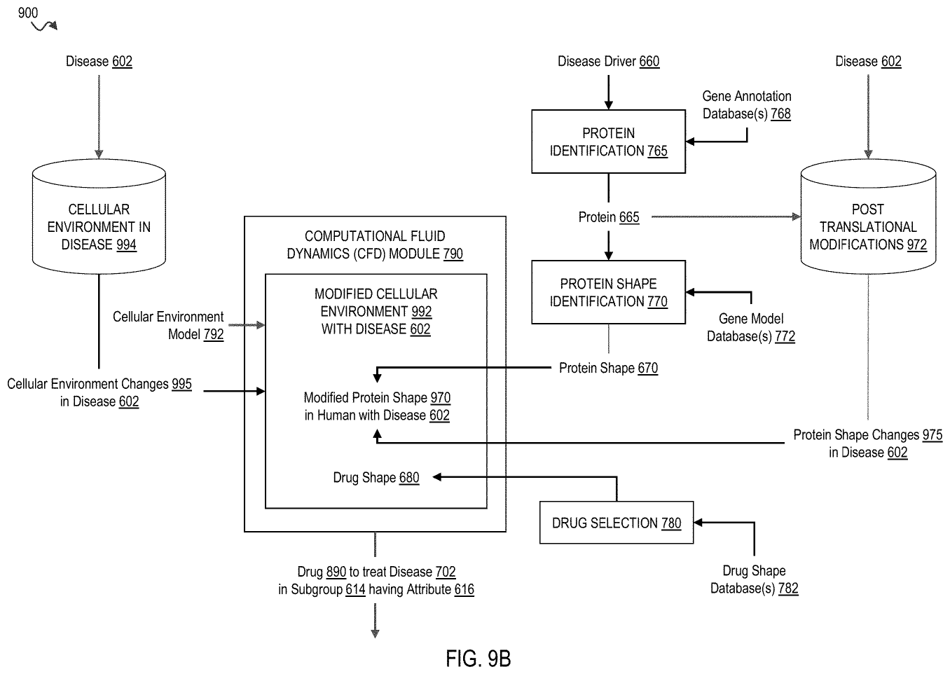

FIGS. 9A and 9B are diagrams illustrating a “protein-drug modeling in disease” process 900 according to an exemplary embodiment.

As shown in FIG. 9A, a natural language processing module 950 is used to analyze published medical research 930 and identify, from that published medical research 930, each indication that a disease 602 causes a change in the cellular environment (cellular environment changes 995). Each disease 602 and each cellular environment change 995 in humans with that disease 602 is stored in a cellular environment in disease database 994.

Similarly, the natural language processing module 950 identifies, in the published medical research 930, each indication that a disease 602 causes a change in the protein shape 670 (protein shape change 976) of a protein 662 in humans with that disease 602. Each disease 602, protein 662 affected by that disease 602, and protein shape change 976 in humans with that disease 602 is stored in a post translational modifications database 972. A graphical user interface 980 may also be provided, enabling researchers to review the published medical research 930 and view and edit the information extracted by the natural language processing module 950 and stored in the cellular environment in disease database 994 and the post translational modifications database 972.

As shown in FIG. 9B, a cellular environment model 892 is provided to the CFD module 790. First, the cellular environment model 892 may more accurately reflect the cellular environment of a healthy human than the conventional salt-water solution. For instance, the cellular environment model 892 may include electrolytes. Additionally, the cellular environment in disease database 994 is searched for entries indicating cellular environment changes 995 in humans with the disease 602. Both the cellular environment model 892 and the cellular environment changes 995 caused by the disease 602 are provided to the CFD module 790 to model the modified cellular environment 992 in humans with the disease 602.

As described above with reference to FIG. 7D, the protein identification module 765 and protein shape identification module 770 identify the protein shape 670 of a protein 665 made by the disease driver 660. Additionally, the post translational modifications database 972 is searched for entries indicating protein shape changes 975 affecting the selected protein 665 in humans with the disease 602. Both the protein shape 670 and the protein shape changes 975 are provided to the CFD module 790 to model the modified protein shape 970 of the selected protein 665 in a human with the disease 602.

By more accurately modeling the modified protein shape 970 and the modified cellular environment 992 in humans with the disease 602, the protein-drug modeling in disease process 900 is better able to identify a drug 890 that will bond with the protein 665 in that modified cellular environment 992.

While a preferred embodiment of the AI-enabled health ecosystem 300 has been described above, those skilled in the art who have reviewed the present disclosure will readily appreciate that other embodiments can be realized within the scope of the invention. Accordingly, the present invention should be construed as limited only by any appended claims.

What is claimed is:

1. A method for personalized, genetics-based drug discovery, the method comprising:
storing medical data that includes physiological data, medical history data, contextual information, and genetics data;
identifying, from the stored medical data, a group of individuals having a disease;
repeatedly partitioning the group of individuals having the disease to select a subgroup of the individuals having a common attribute;
for each selected subgroup:
detecting and storing physiological anomalies or medical test anomalies that are more prevalent in the physiological data or the medical history data of the selected subgroup than in the physiological data or the medical history data of a control group;
performing genetics differential analysis to identify genetic anomalies affecting one or more genes that are more prevalent in the genetics data of the selected subgroup than in the genetics data of the control group;
identifying physiological functions effected by the physiological anomalies or medical test anomalies;
identifying biological functions effected by the genes having the genetic anomalies;
ranking the potential nodal points from among the genes having genetic anomalies that are most likely to have caused the largest number of the identified genetic anomalies in the genetic data of the selected subgroup;
identifying, based on the effected physiological functions and the effected biological functions, the disease driver from among the potential nodal points most likely to have caused of the identified genetic anomalies in the genetic data of the selected subgroup; and
identifying a drug to treat the disease in individuals having the attribute by identifying a drug that binds to a protein made by the disease driver.

2. The method of claim 1, further comprising:
determining whether the selected subgroup has a statistically significant disease signature compared to the control group.

3. The method of claim 1, wherein the group of individuals is partitioned to select a different subgroup having a different attribute in response to a determination that the selected subgroup does not have a statistically significant disease signature compared to the control group.

4. The method of claim 1, wherein the potential nodal points, the disease driver, or the drug to treat the disease in individuals having the attribute is identified in response to a determination that the selected subgroup does not have a statistically significant disease signature compared to the control group.

5. The method of claim 1, wherein the drug that binds to the protein made by the disease driver is identified by using computational fluid dynamics to model cellular conditions, the shape of the protein, and a plurality of drugs.

6. The method of claim 5, wherein modeling the shape of the protein comprises:
storing changes to shapes of a plurality of proteins caused by a plurality of diseases;
identifying at least one change to the shape of the protein caused by the disease; and
using computation fluid dynamics to model the shape of the protein as modified by the at least one change to the shape of the protein caused by the disease.

7. The method of claim 6, wherein modeling cellular conditions comprises:
storing cellular conditions changes caused by a plurality of diseases;
selecting at least one cellular condition change caused by the disease; and
using computation fluid dynamics to model the cellular conditions as modified by the at least one cellular condition change caused by the disease.

8. The method of claim 7, wherein the cellular conditions changes caused by the plurality of diseases and the changes to shapes of a plurality of proteins caused by the plurality of diseases are identified by analyzing published medical research using natural language processing.

9. The method of claim 1, further comprising:
performing genetics differential analysis to identify a genetic anomaly affecting an unannotated genes; and
storing an annotation that the unannotated gene may be related to an effected physiological function of a physiological anomaly or a medical test anomaly.

10. The method of claim 9, further comprising:
identifying a biological function effected by a gene in another animal that is correlated with the unannotated gene.

11. An artificial intelligence-enabled health ecosystem comprising:
non-transitory computer readable storage media that stores medical data that includes physiological data, medical history data, contextual information, and genetics data;
a hardware computer processor that:
identifies, from the stored medical data, a group of individuals having a disease;
repeatedly partitions the group of individuals having the disease to select a subgroup of the individuals having a common attribute;
for each selected subgroup:
detects and stores physiological anomalies or medical test anomalies that are more prevalent in the physiological data or the medical history data of the selected subgroup than in the physiological data or the medical history data of a control group;
performs genetics differential analysis to identify genetic anomalies affecting one or more genes that are more prevalent in the genetics data of the selected subgroup than in the genetics data of the control group;
identifies physiological functions effected by the physiological anomalies or medical test anomalies;
identifies biological functions effected by the genes having the genetic anomalies;
ranks the potential nodal points from among the genes having genetic anomalies that are most likely to have caused the largest number of the identified genetic anomalies in the genetic data of the selected subgroup;
identifies, based on the effected physiological functions and the effected biological functions, the disease driver from among the potential nodal points most likely to have caused of the identified genetic anomalies in the genetic data of the selected subgroup; and
identifies a drug to treat the disease in individuals having the attribute by identifying a drug that binds to a protein made by the disease driver.

12. The system of claim 11, wherein the computer processor is further configured to determine whether the selected subgroup has a statistically significant disease signature compared to the control group.

13. The system of claim 11, wherein the processor is configured to partition the group of individuals to select a different subgroup having a different attribute in response to determination that the selected subgroup does not have a statistically significant disease signature compared to the control group.

14. The system of claim 11, wherein the processor is configured to identify the potential nodal points, the disease driver, or the drug to treat the disease in individuals having the attribute in response to a determination that the selected subgroup has a statistically significant disease signature compared to the control group.

15. The system of claim 11, wherein the processor is configured to identify the drug that binds to the protein by the disease driver by using computational fluid dynamics to model cellular conditions, the shape of the protein, and a plurality of drugs.

16. The system of claim 15, wherein the processor is configured to model the shape of the protein by:
storing changes to shapes of a plurality of proteins caused by a plurality of diseases;
identifying at least one change to the shape of the protein caused by the disease; and
using computation fluid dynamics to model the shape of the protein as modified by the at least one change to the shape of the protein caused by the disease.

17. The system of claim 16, wherein the processor is configured to model cellular conditions by:
storing cellular conditions changes caused by a plurality of diseases;
selecting at least one cellular condition change caused by the disease; and
using computation fluid dynamics to model the cellular conditions as modified by the at least one cellular condition change caused by the disease.

18. The system of claim 17, wherein the cellular conditions changes caused by the plurality of diseases and the changes to shapes of a plurality of proteins caused by the plurality of diseases are identified by analyzing published medical research using natural language processing.

19. The system of claim 11, wherein the processor is further configured to:
perform genetics differential analysis to identify a genetic anomaly affecting an unannotated genes; and
store an annotation that the unannotated gene may be related to an effected physiological function of a physiological anomaly or a medical test anomaly.

20. The system of claim 19, wherein the processor is further configured to identify a biological function effected by a gene in another animal that is correlated with the unannotated gene.
